(19) United States
(12) Patent Application Publication
(10) Pub. No.: US 2004/0202688 A1
(43) Pub. Date: Oct. 14, 2004

(54) Nail varnish composition comprising at least one film-forming gradient copolymer and cosmetic process for making up or caring for the nails

(76) Inventors: Nathalie Mougin, Paris (FR); Colette Cazeneuve, Paris (FR); Marco Vicic, Bry S/Marne (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/734,298

(22) Filed: Dec. 15, 2003

Related U.S. Application Data
(60) Provisional application No. 60/458,002, filed on Mar. 28, 2003.

(30) Foreign Application Priority Data
Dec. 13, 2002 (FR) 02 15857

(51) Int. Cl. A61K 7/00

(52) U.S. Cl. 424/401

(57) ABSTRACT

The present disclosure relates to a nail varnish composition comprising at least one film-forming gradient copolymer exhibiting a mass polydispersity index (PI) of less than or equal to 2.5, the said composition being capable of forming a film exhibiting a rate of loss of weight of less than 1 mg/minute when the film is subjected to abrasion produced with the Taber abrasion tester at 23° C.

The invention also relates to a cosmetic process for making up or caring for the nails.

This application claims benefit of U.S. Provisional Application No. 60/458,002, filed Mar. 28, 2003.

Disclosed herein are novel cosmetic compositions, for instance, make-up compositions, such as nail varnish compositions, comprising at least one film-forming gradient copolymer, for example, amphiphilic gradient copolymers, which can be soluble or dispersible in water and/or in organic solvents, wherein the make-up compositions exhibit good hold properties.

It is known that nail varnish compositions may comprise a film-forming polymer in an organic solvent medium or an aqueous medium. The varnish can form, after drying, a colored or colorless film on the nails and thus can make it possible to embellish the nails or protect the nails against external attack, such as rubbing or scratches. Nail varnishes, however, may frequently exhibit poor hold over time: the film may deteriorate after one or two days, for example, by flaking or detaching. Such a deterioration often occurs at the end of the nail. When the varnish becomes damaged, the user may have to remove the damaged varnish and then carry out a fresh application of varnish. The user can also retouch the damaged varnish by partially applying varnish, but this type of retouching does not normally result in an entirely attractive make-up. If the user does nothing, the damaged varnish may detract from the attractive appearance of the make-up, and does not give good protection to the nail.

Other nail varnishes, such as easily peelable varnishes or varnishes which can be removed with water, may not confer a very good hold over time either.

Furthermore, the use within the same composition of a mixture of several polymers with very different chemical natures, even if each polymer contributes a desired characteristic, can cause problems of phase separation within the composition because the respective chemical natures of the different polymers may not necessarily be compatible.

The use of random polymers, for example of conventional acrylic polymers obtained by conventional radical polymerization by random mixing of monomers, may not allow the problems described above to be solved satisfactorily. This is because the random polymers known in the art may exhibit a dispersity in composition of the polymer chains, which can also result in a phase separation of the polymers within the formula.

A need thus exists to have available nail varnish compositions that can make it possible to obtain a film deposited on the nails which exhibits a satisfactory hold over time, without exhibiting unsightly flaws, and wherein the compositions are, for example, stable and homogeneous.

One aspect of the present disclosure is thus to provide such a nail varnish composition, which can exhibit at least one property chosen from good stability, and good properties of hold over time, such as good resistance to rubbing, water and/or to flaking.

Disclosed herein is therefore a nail varnish composition comprising, in a cosmetically acceptable medium, at least one film-forming gradient copolymer comprising at least two different monomeric units and wherein the at least one film-forming gradient copolymer can exhibit a mass polydispersity index (PI) of less than or equal to 2.5, such as, ranging from 1.1 to 2.3, for example, ranging from 1.15 to 2.0, or, for further example, ranging from 1.2 to 1.9, and further wherein the composition is capable of forming a film that exhibits a rate of loss of weight of less than 1 mg/minute, for example, less than 0.8 mg/minute, and such as less than 0.6 mg/minute, or even less than 0.4 mg/minute, when the film is subjected to abrasion produced with the Taber abrasion tester at 23° C.

As used in present disclosure, the term “film-forming polymer” is understood to mean a polymer capable of forming, by itself alone or in the presence of an additional agent that is able to form a film, a continuous film which adheres to the nail at a temperature ranging from 20° C. to 30° C.

The at least one copolymer according to the present disclosure is chosen from gradient copolymers, which comprise at least two different monomeric units, and which exhibit a low polydispersity in mass and can also exhibit a low polydispersity in composition.

As disclosed herein, the at least one film-forming gradient copolymer exhibits a low dispersity in composition, i.e., all the chains exhibiting virtually the same structures, and the copolymers are therefore compatible with one another. Without being bound by theory, the result of this low dispersity in composition is that the cosmetic compositions comprising these copolymers do not exhibit the disadvantages and limitations of the compositions of the prior art.

For example, the film-forming gradient copolymers, as disclosed herein, exhibit the useful property of being easy to handle in water or in an organic solvent medium while retaining beneficial Theological properties.

The polydispersity in mass can be illustrated using the mass polydispersity index (PI) of the copolymer, which is equal to the ratio of the weight-average molecular mass (Mw) to the number-average molecular mass (Mn). A low dispersity in mass reflects approximately identical chain lengths, which is the case for the copolymers according to the present disclosure.

The at least one film-forming gradient copolymer according to the disclosure has a mass polydispersity index of less than or equal to 2.5, for example, a mass polydispersity index ranging from 1.1 to 2.3, such as ranging from 1.15 to 2.0, or even ranging from 1.2 to 1.9.

Further, the weight-average molecular mass of the at least one film-forming gradient copolymer can range from 5 000 g/mol to 1 000 000 g/mol, for example, from 500 g/mol to 800 000 g/mol, such as from 6 000 g/mol to 500 000 g/mol.

The number-average molecular mass of the at least one film-forming gradient copolymer can range from 5 000 g/mol to 1 000 000 g/mol, for instance, from 5 500 g/mol to 800 000 g/mol, and for further example, from 6 000 g/mol to 500 000 g/mol.

The weight-average molecular masses (Mw) and the number-average molecular masses (Mn) are determined by gel permeation liquid chromatography (GPC), with the eluent THF, a calibration curve established with linear polystyrene standards, and a refractometer detector.

The at least one film-forming gradient copolymer disclosed herein can also exhibit a low dispersity in composition, which means that all the chains of copolymers have a composition (i.e., a sequence of monomeric units) that is approximately the same, and are therefore basically homogeneous in composition.

In order to show that all the chains of copolymers have a similar composition, use can be made of liquid adsorption chromatography (LAC), which makes it possible to separate the chains of copolymers, not according to their molecular weight, but according to their polarity. The polarity reflects the chemical composition of the polymers constituting the material, the monomers being known. Reference may be made to the publication Macromolecules (2001), 34, 2667, which describes the LAC technique.

The polydispersity in composition can be defined for example, from the LAC curve (i.e., the curve representing the proportion of polymers as a function of the elution volume): if “V^{1/2} min” is used to denote the minimum value of the elution volume at mid-height of the curve, and if “V^{1/2} max” is used to denote the maximum value of the elution volume at mid-height of the curve, the polydispersity in composition can be considered low if the difference (V^{1/2} max−V^{1/2} min) is less than or equal to 3.5, for example, ranging from 1 to 2.8, such as ranging from 1.2 to 2.5.

Furthermore, the LAC curve can also be defined by a Gaussian curve of formula:

$$y = \frac{A}{W \sqrt{\frac{π}{2}}} \times {ⅇ}^{- 2 \frac{{( x - {x}_{0} )}^{2}}{{W}^{2}}} + {y}_{o}$$

wherein:

x₀ is the value of x (elution volume) at the center of the peak

w is equal to twice the standard deviation of the Gaussian distribution (i.e. 2σ), or alternatively corresponds to approximately 0.849 times the width of the peak at mid-height

A is equal to the area under the peak

yₒ is the value of y corresponding to x₀.

The dispersity in composition can also be defined by the value w as defined above. For example, the value w can range from 1 to 3, such as from 1.1 to 2.3, and for further example, from 1.1 to 2.0.

The gradient copolymers according to the present disclosure can be obtained by living or pseudo-living polymerization.

It is known that living polymerization is a polymerization in which the growth of the polymer chains only stops when the monomeric unit disappears. The number-average molecular mass (Mn) increases with the conversion. Anionic polymerization is a typical example of living polymerization. Such polymerization may result in copolymers having a low dispersity in mass, that is to say, polymers with a mass polydispersity index (PI) generally of less than 2.

As for pseudo-living polymerization, it is generally associated with controlled radical polymerization. Non-limiting mention may be made, among the main types of controlled radical polymerization, of:

radical polymerization controlled by nitroxides. For example, reference may be made to Patent Applications Nos. WO 96/24620 and WO 00/71501, which disclose the tools of this polymerization and their use, and to the papers published by Fischer (Chemical Reviews, 2001, 101, 3581), by Tordo and Gnanou (J. Am. Chem. Soc., 2000, 122, 5929) and by Hawker (J. Am. Chem. Soc., 1999, 121, 3904);

atom transfer radical polymerization, disclosed for example, in Patent Application No. WO 96/30421 and which proceeds by the reversible insertion of an organometallic complex in a bond of carbon-halogen type;

radical polymerization controlled by sulphur derivatives chosen from xanthate, dithioester, trithiocarbonate and carbamate types, such as disclosed in Patent Applications Nos. FR 2 821 620, WO 98/01478, WO 99/35177, WO 98/58974, WO 99/31144 and WO 97/01478, and in the publication by Rizzardo et al. (Macromolecules, 1998, 31, 5559).

Controlled radical polymerization denotes polymerizations wherein the secondary reactions, which usually result in the disappearance of propagating entities, (e.g., via termination or transfer reaction) are rendered highly improbable in comparison with the propagation reaction, by virtue of an agent for controlling the free radicals. A disadvantage of this method of polymerization can be that, when the concentrations of free radicals become high in comparison with the concentration of the monomer, the secondary reactions again become determining and tend to broaden the distribution of the masses.

By virtue of the polymerization methods, the polymer chains of the gradient copolymers as disclosed herein, grow simultaneously, and therefore incorporate at each instant the same ratio of comonomers. Thus, the polymer chains have basically the same structures or similar structures, resulting in a low dispersity in composition. These chains also have a low mass polydispersity index.

Gradient copolymers are copolymers exhibiting a change in the ratio of the various monomeric units all along the chain. The distribution in the polymeric chains of the comonomers depending on the change during the synthesis of the relative concentrations of the comonomers.

The film-forming gradient copolymers according to the disclosure comprise at least two different monomeric units, wherein the concentration of the at least two different monomeric units along the polymer chain changes gradually, and in a systematic and predictable way. Accordingly, the polymer chains have at least one monomeric unit Mi, wherein, whatever the normalized position x on the polymer chain, there is a non-zero probability of encountering the monomeric unit Mi along each chain.

One of the characteristics that defines gradient copolymers is that, at any instant in the polymerization, all the chains are subjected to the presence of the combination of all the monomeric units. Thus, in the reaction medium, the concentration of each monomeric unit is always non-zero at any instant in the polymerization. This characteristic helps distinguish the copolymers according to the present disclosure, from conventional block polymers in which the change in the monomeric units along the polymer chain is not systematic. For example, for an AB diblock polymer, within the A block, the concentration of the other monomeric unit B is always zero.

In the case of random polymers, the change in the monomeric units along the polymer chain will not be gradual, systematic or predictable either. As illustrated by the diagram below, a random polymer obtained by conventional radical polymerization of two monomeric units is distinguished from a gradient copolymer by the distribution of the monomeric units, which is not identical over all the chains for the random polymer, and by the length of the chains, which is not identical for all the chains of the random polymer.

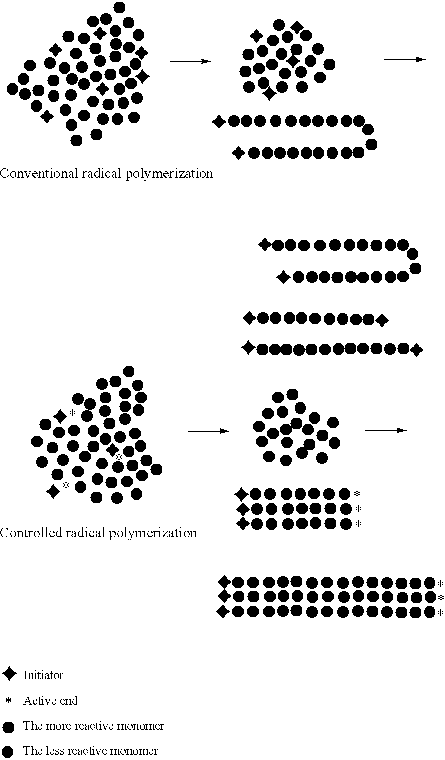

For a theoretical description of gradient copolymers, reference may be made to the following publications:

T. Pakula et al., Macromol. Theory Simul., 5, 987-1006 (1996);

A. Aksimetiev et al., J. of Chem. Physics, 111, No. 5;

M. Janco, J. Polym. Sci., Part A: Polym. Chem. (2000), 38(15), 2767-2778;

M. Zaremski et al., Macromolecules (2000), 33(12), 4365-4372;

K. Matyjaszewski et al., J. Phys. Org. Chem. (2000), 13(12), 775-786;

Gray, Polym. Prepr. (Am. Chem. Soc., Div. Polym. Chem.) (2001), 42(2), 337-338;

K. Matyjaszewski, Chem. Rev. (Washington, D.C.) (2001), 101(9), 2921-2990.

Among gradient copolymers, it is possible to distinguish between natural gradient copolymers and artificial gradient copolymers.

A natural gradient copolymer is a gradient copolymer synthesized as a batch from a starting mixture of the comonomers. The distribution in the chain of the various monomeric units follows a pattern deduced from the relative reactivity, and from the starting concentrations, of the monomeric units. These copolymers constitute the simplest class of gradient copolymers as it is the starting mixture which defines the final product.

An artificial gradient copolymer is a gradient copolymer for which the concentration of monomeric units during the synthesis is varied by a processing expedient. In the case of artificial gradient copolymers, the mixture of monomeric units in the chain is changed to another mixture of monomeric units in the chain due to a sudden and abrupt change in the monomeric units in the reaction medium (for instance, via stripping of the first mixture, or addition of at least one new monomeric unit). It is even possible for at least one monomeric unit therein to completely disappear, to the benefit of at least one other monomeric unit.

The gradient can be characterized experimentally by measuring, during polymerization, the chemical composition of the polymer. This measurement is performed indirectly by determining the change in the content of the various monomeric units at any instant. It can be performed by NMR and UV spectroscopy, for example.

Without being bound by theory, it is believed that for the polymers prepared by living or pseudo-living polymerization, the length of the chains is linearly related to the conversion. By withdrawing a sample of the polymerization solution at various instants during the polymerization, and by measuring the difference in content of each monomeric unit, the composition of the gradient is thus determined.

In gradient copolymers, the distribution of the compositions of the chains is narrow. For example, there is no overlap between the peak of the gradient copolymer, and those of the respective homopolymers. In other words, the copolymer obtained under gradient conditions is composed of polymer chains with the same composition whereas, in conventional random polymerization, chains with different compositions coexist, including those of the respective homopolymers.

It is also possible to characterize gradient copolymers by a vector characteristic of each copolymer.

Since it is assumed that there exists an infinite number of polymers characterized by a given chemical composition, to specify a polymer it is possible to describe the distribution of monomeric units along the chain. This involves a description comprising several variables. The vector is a point of the space of the chemical compositions.

The exact term is that G is a vector, wherein the coordinates of the vector are the concentrations of the monomeric units along the polymer chain. The concentrations are defined by the rules of the reactivity coefficients of each of the monomeric units and therefore are related to the concentration of the free monomeric units during the synthesis: from the moment that the monomeric unit is not in zero concentration in the reaction mixture, it is not in zero concentration in the polymer.

It is therefore possible to characterize gradient copolymers by the function G(x) which defines the composition gradient:

\vec{G}(x)=Σ\vec{[Mi](x)}

wherein:

x is a normalized position on the polymer chain and

[Mi](x) is the relative concentration, in this position x, of the monomeric unit Mi, expressed in mol %.

The function G(x) therefore locally describes the composition of the gradient copolymer.

Two copolymers can have an equivalent composition overall but very different local distributions of the monomeric units and therefore different gradients. For example, in the case of a (50/50) AB diblock copolymer, the function [A] has a value of 1 up to x=½, and then 0 subsequently.

The factors that can determine the gradient include: the relative reactivity coefficients of each monomeric unit (referred to as rᵢ for the monomeric unit Mi), which depend mainly on the type of synthesis process employed (e.g., homogeneous, dispersed) and on the solvents used; the starting concentrations of each of the monomeric units, and the possible additions of monomeric units during the polymerization.

Thus, for example, if a gradient copolymer comprising styrene (M1) having a relative reactivity coefficient r₁=0.418 and methacrylic acid (M2), with r₂=0.6, in a homogeneous polymerization system is considered, the variation in the starting concentrations of styrene and methacrylic acid makes it possible to obtain different gradient copolymers having chains with completely different structures. When the starting concentration of methacrylic acid is 10% by weight, a very weak gradient copolymer is obtained in the end for which nanostructure formation cannot be expected. When the starting concentration is 20% by weight, a gradient copolymer is obtained which has a hydrophilic “head” and a hydrophobic “tail” with a sufficiently pronounced gradient to result in nanostructure formation. When the starting concentration is 50% by weight, since the aforementioned monomeric units are isoreactive under these conditions, the copolymer obtained is of alternating type.

Although the copolymers described are all gradient copolymers of styrene and of methacrylic acid, the difference in starting concentration of the monomeric units results in chains with completely different structures, which confer different properties on the copolymers. The example therefore illustrates the effect that the starting monomeric unit has on the arrangement along the chain of the various monomeric units.

In the case of a styrene/methacrylic acid gradient copolymer, the various polymers obtained can be represented diagrammatically, as below, wherein the white units correspond to styrene and the dark units correspond to methacrylic acid:

10% of methacrylic acid Initially:

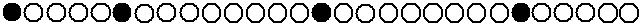

Very weak gradient copolymer for which nanostructure formation cannot be expected.

20% of methacrylic acid Initially:

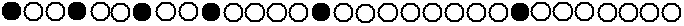

Copolymer with a hydrophilic “head” and hydrophobic “tail” with a sufficiently pronounced gradient to result in nanostructure formation.

50% of methacrylic acid Initially:

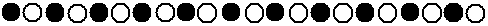

As the monomeric units are isoreactive under these conditions, the copolymer obtained is of the alternating type.

The structure of these copolymers can be determined by the disappearance of the methacrylic acid as a function of the degree of conversion.

As disclosed herein, the at least one film-forming gradient copolymer comprises at least two different monomeric units, wherein each monomeric unit can be present in an amount ranging from 1 to 99% by weight, relative to the weight of the final copolymer, for example, in an amount ranging from 2 to 98% by weight, relative to the weight of the final copolymer, such as in an amount ranging from 5 to 95% by weight, relative to the weight of the final copolymer.

In one aspect of the present disclosure, the at least one film-forming gradient copolymer comprises at least one hydrophilic monomeric unit.

The at least one hydrophilic monomeric unit can be present in the copolymer in an amount ranging from 1 to 99% by weight, relative to the total weight of the copolymer, for example, ranging from 2 to 70% by weight, relative to the total weight of the copolymer, such as ranging from 3 to 50% by weight, relative to the total weight of the copolymer, or for instance, ranging from 4 to 30% by weight, relative to the total weight of the copolymer, and for further example, ranging from 5 to 25% by weight, relative to the total weight of the copolymer.

As used in the present disclosure, the term “hydrophilic monomeric unit” will denote without distinction monomeric units whose homopolymers are soluble or dispersible in water, or whose ionic form is soluble or dispersible in water.

A homopolymer is “water-soluble” if it forms a clear solution when it is in solution in water at 1% by weight, at 25° C.

A homopolymer is “water-dispersible” if, in water at 1% by weight, at 25° C., it forms a stable suspension of fine particles, for example, spherical particles. The mean size of the particles comprising the dispersion is less than 1 μm and can range from 5 to 400 nm, for example, from 10 to 250 nm, wherein the particle sizes are measured by light scattering.

In one aspect of the present disclosure, the homopolymer(s) formed from the at least one hydrophilic monomeric unit can have a Tg of greater than or equal to 20° C., for example, greater than or equal to 50° C.

In another aspect of the present disclosure, the homopolymer(s) formed from the at least one hydrophilic monomeric unit can optionally have a Tg of less than or equal to 20° C.

In yet another aspect of the present disclosure, the at least one film-forming gradient copolymer comprises at least one hydrophobic monomeric unit, such as at least one hydrophobic monomeric unit capable of being rendered hydrophilic after polymerization. The at least one hydrophobic monomeric unit can be rendered hydrophilic, for example, by chemical reaction, such as hydrolysis, or by chemical modification, for example, chemical modification of an ester functional group, by incorporation of chains comprising a hydrophilic unit, for example, of carboxylic acid type.

The at least one hydrophobic monomeric unit can be present in the copolymer in an amount ranging from 1 to 99% by weight, relative to the total weight of the copolymer, for example, in an amount ranging from 30 to 98% by weight, relative to the total weight of the copolymer, such as, in an amount ranging from 50 to 97% by weight, relative to the total weight of the copolymer, and, for instance ranging from 70 to 96% by weight, relative to the total weight of the copolymer, and for further example, ranging from 75 to 95% by weight, relative to the total weight of the copolymer.

According to one aspect of the disclosure, the homopolymer(s) formed from the at least one hydrophobic monomeric unit has a Tg greater than or equal to 20° C., such as greater than or equal to 30° C.

According to another aspect of the disclosure, the homopolymer(s) formed from the at least one hydrophobic monomeric unit has a Tg of less than or equal to 20° C.

Thus, according to still another aspect of the present disclosure, the at least one film-forming gradient copolymer as disclosed herein comprises at least one monomeric unit, the homopolymer of which has a Tg of less than or equal to 20° C., for example, a Tg ranging from −150° C. to 20° C., such as ranging from −130° C. to 18° C., and for futher example, ranging from −120° C. to 15° C.

The at least one monomeric unit, the homopolymer of which has a Tg less than or equal to 20° C. can be present in the copolymer in an amount ranging from 1 to 99% by weight, relative to the total weight of the copolymer, for example, ranging from 20 to 90% by weight, relative to the total weight of the copolymer, such as ranging from 30 to 85% by weight, relative to the total weight of the copolymer, and for instance, ranging from 50 to 75% by weight, relative to the total weight of the copolymer.

The at least one monomeric unit, the homopolymer of which has a Tg greater than or equal to 20° C. can be present in the copolymer in an amount ranging from 1 to 99% by weight, relative to the total weight of the copolymer, for example, ranging from 10 to 80% by weight, relative to the total weight of the copolymer, such as ranging from 15 to 70% by weight, relative to the total weight of the copolymer, and further for example, ranging from 25 to 50% by weight, relative to the total weight of the copolymer.

As used in the present disclosure, the term “monomeric unit with a Tg” will denote the monomeric units whose homopolymer has such a Tg, measured according to the method described below.

According to the present disclosure, the Tg (or glass transition temperature) is measured according to Standard ASTM D3418-97 by differential scanning calorimetry (DSC) on a calorimeter over a temperature range from −100° C. to +150° C., at a heating rate of 1° C./min in aluminium crucibles with a capacity of 150 μl.

In one aspect of the present disclosure, the at least one film-forming gradient copolymer as disclosed herein, comprises three different monomeric units, which can each be present in the copolymer in an amount ranging from 5 to 90% by weight, relative to the total weight of the copolymer, for example, ranging from 7 to 86% by weight, relative to the total weight of the copolymer.

For example, the at least one film-forming gradient copolymer can comprise a first monomeric unit in an amount ranging from 5 to 25% by weight, relative to the total weight of the copolymer, a second monomeric unit in an amount ranging from 5 to 25% by weight, relative to the total weight of the copolymer, and a third monomeric unit in an amount ranging from 50 to 90% by weight, relative to the total weight of the copolymer.

According to one aspect of the disclosure, the at least one film-forming gradient copolymer as disclosed herein can comprise a hydrophilic monomeric unit in an amount ranging from 5 to 25% by weight relative to the total weight of the copolymer, a monomeric unit, the homopolymer of which has a Tg of less than or equal to 20° C. in an amount ranging from 50 to 90% by weight relative to the total weight of the copolymer, and an additional monomeric unit in an amount ranging from 5 to 25% by weight, relative to the total weight of the copolymer.

Among the hydrophilic monomeric units capable of being used as disclosed herein, non-limiting mention may be made of the following monomeric units:

amino(C₁-C₄ alkyl) (meth)acrylate derivatives, for instance N,N-di(C₁-C₄ alkyl)amino(C₁-C₆ alkyl)(meth)acrylates, such as N,N-dimethylaminoethyl methacrylate (MADAME) and N,N-diethylaminoethyl methacrylate (DEAMEA);

N,N-di(C₁-C₄ alkyl)(meth)acrylamides and N,N-di(C₁-C₄ alkyl)amino(C₁-C₆ alkyl)(meth)acrylamides, such as N,N-dimethylacrylamide, N,N-dimethylaminopropylacrylamide (DMAPA) and N,N-dimethylaminopropylmethacrylamide (DMAPMA),

di(C₁-C₈ alkyl)allylamines, such as dimethyldiallylamine;

vinylamine;

vinylpyridines, for example, 2-vinylpyridine and 4-vinylpyridine; and the acid salts and quaternized forms thereof.

Non-limiting mention may be made, among inorganic acids, of sulphuric acid, hydrochloric acid, hydrobromic acid, hydriodic acid, phosphoric acid and boric acid.

Non-limiting mention may be made, among organic acids, of acids comprising at least one group chosen from carboxyl, sulpho and phosphono groups. The acids can be chosen from linear, branched, cyclicaliphatic and aromatic acids. The acids can additionally comprise at least one heteroatom chosen from O and N, for example in the form of hydroxyl groups. Examples of an acid with an alkyl group include acetic acid CH₃COOH and propionic acid. An example of a polyacid is terephthalic acid. Examples of hydroxyacids include citric acid and tartaric acid.

The quaternizing agents can chosen from alkyl halides, such as methyl bromide, alkyl sulphates, such as methyl sulphate, and propane sultones.

Among the hydrophilic monomeric units as disclosed herein, non-limiting mention may also be made of:

ethylenic carboxylic acids, for instance, mono- and dicarboxylic acids, such as acrylic acid, methacrylic acid, crotonic acid, itaconic acid, fumaric acid and maleic acid;

carboxylic anhydrides carrying a vinyl bond, such as maleic anhydride;

ethylenic sulphonic acids, such as styrenesulphonic acid, acrylamidopropanesulphonic acid, and the salts thereof;

vinylbenzoic acid, vinylphosphonic acid and the salts thereof.

the potassium salt of 3-(acryloyloxy)propanesulphonic acid and the compound of formula CH₂═CHCOOCH₂OCH₂(OH)CH₂SO₃⁻Na⁺.

The neutralizing agent can be chosen from inorganic bases, such as LiOH, NaOH, KOH, Ca(OH)₂ and NH₄OH; and organic bases, for example a primary, secondary or tertiary amine, for instance, an optionally hydroxylated alkylamine, such as dibutylamine, triethylamine and stearamine, and 2-amino-2-methylpropanol, monoethanolamine, diethanolamine and stearamidopropyldimethylamine.

Among the hydrophilic monomeric units as disclosed herein, further, non-limiting mention may also be made of:

amides of unsaturated carboxylic acids, such as acrylamides, methacrylamides, and their N-substituted derivatives, for example N-(C₁-C₄ alkyl)(meth)acrylamides, such as N-methylacrylamide, and N,N-di(C₁-C₄ alkyl)(meth)acrylamides, such as N,N-dimethylacrylamide;

hydroxyalkyl (meth)acrylates, for example, those in which the alkyl group comprises from 2 to 4 carbon atoms, such as hydroxyethyl (meth)acrylate;

(meth)acrylates of polyethylene glycol (5 to 100 EO) and of glycol, which may optionally be substituted on their end functional group by a group chosen from alkyls, phosphates, phosphonates and sulphonates, for example glyceryl acrylate, methoxypolyethylene glycol (8 or 12 EO) (meth)acrylate, and hydroxypolyethylene glycol (meth)acrylate;

alkoxyalkyl (meth)acrylates, such as ethoxyethyl (meth)acrylate;

polysaccharide (meth)acrylates, such as sucrose acrylate;

vinylamides, such as vinylacetamide; optionally cyclic vinylamides, for example, vinyllactams, such as N-vinylpyrrolidone and N-vinylcaprolactam;

vinyl ethers, such as vinyl methyl ether.

Additionally, among the hydrophilic monomeric units as disclosed herein, non-limiting mention may also be made of:

methacrylamidopropoxytrimethylammonium betaines;

N,N-dimethyl-N-methacryloyloxyethyl-N-(3-sulphopropyl)ammonium betaines;

3-methacryloylethoxycarbonylpyridiniums;

the compounds of formula:

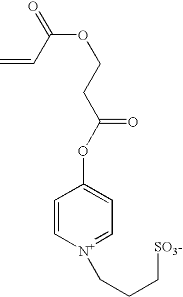

and 4-vinylpyridiniumsulphopropyl betaines of formula:

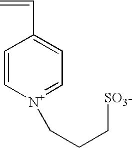

According to one aspect of the present disclosure, the hydrophilic monomeric units are chosen from units of N,N-dimethylaminoethyl methacrylate (MADAME), acrylic acid, methacrylic acids crotonic acid, styrenesulphonic acid, acrylamidopropanesulphonic acid, dimethylaminopropylmethacrylamide (DMAPMA), styrenesulphonate, hydroxyethyl acrylate, glyceryl acrylate, ethoxyethyl methacrylate, ethoxyethyl acrylate, methoxypolyethylene glycol (8 and 12 EO) (meth)acrylate, hydroxypolyethylene glycol (meth)acrylate, N-vinylpyrrolidone, N-vinylcaprolactam, acrylamide and N,N-dimethylacrylamide

Among hydrophobic monomeric units capable of being rendered hydrophilic, such as by hydrolysis, non-limiting mention may be made of: C₁-C₄ alkyl (meth)acrylates, such as tert-butyl (meth)acrylates and ethyl (meth)acrylates, which result in the production of (meth)acrylic acids after hydrolysis.

Among monomeric units, as disclosed herein, whose homopolymer has a Tg of less than or equal to 20° C., some of which may be hydrophilic, non-limiting mention may be made of:

ethylenic hydrocarbons comprising from 2 to 10 carbons, such as ethylene, isoprene and butadiene;

acrylates of formula CH₂═CHCOOR₁, wherein R₁ is chosen from saturated and unsaturated, linear and branched, hydrocarbon groups comprising from 1 to 12 carbons, with the exception of the tert-butyl group, optionally comprising at least one heteroatoms chosen from O, N, S and Si, and further wherein the hydrocarbon group is optionally substituted by at least one substituent chosen from hydroxyl groups and halogen atoms chosen from Cl, Br, I and F atoms;

non-limiting examples of R₁ include: methyl, ethyl, propyl, butyl, isobutyl, hexyl, ethylhexyl, octyl, lauryl, isooctyl, isodecyl, hydroxyethyl, hydroxypropyl, methoxyethyl, ethoxyethyl, methoxypropyl, ethylperfluorooctyl and propylpolydimethylsiloxane groups;

R₁ can also be chosen from groups of formula: —(R″)ₓ—(OC₂H₄)ₙ—OR′″, wherein

x is an integer equal to 0 or 1,

R″ is chosen from saturated and unsaturated, linear and branched, hydrocarbon groups comprising from 1 to 12 carbons,

n is an integer ranging from 5 to 100, and

R′″ is chosen from H and CH₃,

for example, R₁ can be chosen from methoxy(PEO)₈-stearyl groups;

methacrylates of formula: CH₂═C(CH₃)—COOR₂

wherein,

R₂ is chosen from saturated and unsaturated, linear and branched, hydrocarbon groups comprising 3 to 12 carbons, and optionally comprising at least one heteroatom chosen from O, N, S and Si, and further wherein the hydrocarbon group can optionally be substituted by at least one substituent chosen from hydroxyl groups and halogen atoms chosen from Cl, Br, I and F atoms;

non-limiting examples of R₂ groups include: hexyl, ethylhexyl, octyl, lauryl, isooctyl, isodecyl, dodecyl, methoxyethyl, methoxypropyl, ethoxyethyl, ethylperfluorooctyl and propylpolydimethylsiloxane groups;

R₂ can also be chosen from groups of formula: —(R″)ₓ—(OC₂H₄)ₙ—OR′″, wherein

x is an integer equal to 0 or 1,

R″ is chosen from saturated and unsaturated, linear and branched hydrocarbon groups comprising 1 to 12 carbons,

n is an integer ranging from 5 to 100, and

R′″ is chosen from H and CH₃,

for example, R₂ can be chosen from methoxy(PEO)₈-stearyl groups;

N- and N,N-substituted derivatives of unsaturated C₁-C₁₂ carboxylic acid amides, for instance, N-(C₁₋₁₂ alkyl)(meth)acrylamides, such as N-octylacrylamide;

vinyl esters of formula: R₃—CO—O—CH═CH₂, wherein R₃ is chosen from linear and branched alkyl groups comprising 2 to 12 carbons, such as vinyl propionate, vinyl butyrate, vinyl ethylhexanoate, vinyl neononanoate and vinyl neododecanoate;

vinyl alkyl ethers, wherein the alkyl comprises 1 to 12 carbons, such as methyl vinyl ether and ethyl vinyl ether.

According to another aspect of the present invention, the monomeric units whose homopolymer(s) has a Tg of less than or equal to 20° C. can be chosen from:

isoprene and butadiene;

methyl acrylates, ethyl acrylates, isobutyl acrylates, n-butyl acrylates, ethylhexyl acrylates, methoxyethyl acrylates, ethoxyethyl acrylates and hydroxypolyethylene glycol acrylates;

ethoxyethyl methacrylates, hexyl methacrylates, ethylhexyl methacrylates and hydroxypolyethylene glycol methacrylates;

N-(C₆-C₁₂ alkyl)(meth)acrylamides, such as N-octylacrylamide;

vinyl esters of formula: R₃—CO—O—CH═CH₂, wherein

R₃ is chosen from linear and branched alkyl groups comprising 6 to 12 carbons, for example, vinyl neononanoate and vinyl neododecanoate.

Among monomeric units as disclosed herein, whose homopolymer has a Tg of greater than or equal to 20° C., some of which may be hydrophilic, non-limiting mention may be made of:

vinyl compounds of formula: CH₂═CH—R₄, wherein

R₄ is chosen from a hydroxyl group; an —NH—C(O)—CH₃ group; an —OC(O)—CH₃ group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group, such as a furfuryl group; wherein the cycloalkyl, aryl, aralkyl, heterocyclic and heterocyclylalkyl groups can optionally be substituted by at least one substituent chosen from hydroxyl groups, halogen atoms and linear and branched C₁ to C₄ alkyl groups, wherein the linear and branched C₁ to C₄ alkyl groups optionally comprise at least one heteroatom chosen from O, N, S and P, and further wherein the said alkyl groups may be optionally substituted by at least one substituent chosen from hydroxyl groups, from halogens (such as Cl, Br, I and F) and from Si atoms. Non-limiting examples of vinyl monomeric units are vinylcyclohexane, styrene and vinyl acetate;

acrylates of formula CH₂═CH—COOR₅, wherein

R₅ is chosen from a tert-butyl group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group, such as furfuryl groups; wherein the cycloalkyl, aryl, aralkyl, heterocyclic and heterocyclylalkyl groups can optionally be substituted by at least one substituent chosen from hydroxyl groups, halogen atoms, and linear and branched C₁ to C₄ alkyl groups, wherein the linear and branched C₁ to C₄ alkyl groups optionally comprise at least one heteroatom chosen from O, N, S and P, and further wherein the linear and branched C₁ to C₄ alkyl groups may be optionally substituted by at least one substituent chosen from hydroxyl groups, from halogens (such as Cl, Br, I and F) and from Si atoms. Non-limiting examples of acrylate monomers include: t-butylcyclohexyl acrylates, tert-butyl acrylates, t-butylbenzyl acrylates, furfuryl acrylates and isobornyl acrylates;

methacrylates of formula CH₂═C(CH₃)—COOR₆, wherein

R₆ is chosen from linear and branched C₁ to C₄ alkyl groups, such as methyl, ethyl, propyl and isobutyl groups, wherein the linear and branched C₁ to C₄ alkyl groups may optionally be substituted by at least one substituent chosen from hydroxyl groups, from halogens (such as Cl, Br, I and F) and from Si atoms; C₃ to C₈ cycloalkyl groups; C₆ to C₂₀ aryl groups; C₇ to C₃₀ aralkyl groups (C₁ to C₄ alkyl group); 4- to 12-membered heterocyclic groups comprising at least one heteroatom chosen from O, N and S; and heterocyclylalkyl (C₁ to C₄ alkyl) groups, such as a furfuryl group; wherein the cycloalkyl, aryl, aralkyl, heterocyclic and heterocyclylalkyl groups may optionally be substituted by at least one substituent chosen from hydroxyl groups, halogen atoms, and linear and branched C₁ to C₄ alkyl groups, wherein the linear and branched C₁ to C₄ alkyl groups may optionally comprise at least one heteroatom chosen from O, N, S and P, and further wherein the linear and branched C₁ to C₄ alkyl groups can optionally be substituted by at least one substituent chosen from hydroxyl groups and halogen atoms (such as Cl, Br, I and F).

Non-limiting examples of methacrylate monomers include: methyl methacrylates, ethyl methacrylates, n-butyl methacrylates, isobutyl methacrylates, t-butylcyclohexyl methacrylates, t-butylbenzyl methacrylates, methoxyethyl methacrylates, methoxypropyl methacrylates and isobornyl methacrylates;

(meth)acrylamides of formula: CH₂═C(R′)—CO—NR₇R₈, wherein

R₇ and R₈, which may be identical or different, are chosen from hydrogen atoms and linear and branched alkyl groups comprising 1 to 12 carbon atoms, such as n-butyl, t-butyl, isopropyl, isohexyl, isooctyl, or isononyl groups, and

R′ is chosen from hydrogen atoms and methyl.

Non-limiting examples of (meth)acrylamide monomers include: N-butylacrylamides, N-(t-butyl)acrylamides, N-isopropylacrylamides, N,N-dimethylacrylamides and N,N-dibutyl-acrylamides.

Among the monomeric units with a Tg of greater than or equal to 20° C., non-limiting mention may be made of:

furfuryl acrylate, isobornyl acrylate, tert-butyl acrylate, tert-butylcyclohexyl acrylate and tert-butylbenzyl acrylate;

methyl methacrylate, n-butyl methacrylate, ethyl methacrylate and isobutyl methacrylate;

styrene and styrenesulphonate;

vinyl acetate and vinylcyclohexane.

A person of ordinary skill in the art will know how to choose the monomeric units and their respective amounts according to the results desired, using both general knowledge in the art and his or her knowledge of the relative reactivity of each monomeric unit. Thus, if a gradient copolymer having hydrophilic monomeric units in the center of a polymer chain is desired, a difunctional initiator and a mixture of monomeric units can be chosen such that the reactivity of the hydrophilic monomeric units is greater than that of the other monomeric units.

Furthermore, it has been found that the preparation processes employed make it possible to adjust and modify the Tg value or values of the gradient copolymer and thus to obtain a gradient copolymer having at least one Tg value as disclosed herein.

The gradient copolymers of the present disclosure can be prepared by a person of ordinary skill in the art according to the following procedure:

1) A mixture of the various monomeric units is prepared, optionally in a solvent, for example, in a stirred reactor. A radical polymerization initiator and an agent for controlling the polymerization are added. The mixture can be placed under a gas atmosphere that is inert with respect to radical polymerization, such as nitrogen and argon.

Non-limiting examples of optional polymerization solvents include: alkyl acetates, such as butyl acetate and ethyl acetate, aromatic solvents, such as toluene, ketone solvents, such as methyl ethyl ketone, and alcohols, such as ethanol. When the mixture of monomeric units is miscible with water, then water can be used as solvent or cosolvent.

2) The mixture is brought to the desired polymerization temperature, with stirring. This temperature is generally chosen within a range from 10° C. to 160° C., such as from 25° C. to 130° C.

The choice of the polymerization temperature is normally chosen according to the chemical composition of the mixture of monomeric units. Thus, for example, monomers having high propagation kinetic constants and a weaker affinity for the control agent will generally be polymerized at a lower temperature (for instance, in the case of a high proportion of methacrylic derivatives, polymerization can be at a temperature ranging 250 from 80° C.).

3) The polymerisation medium may optionally be modified during the polymerization, before 90% conversion of the starting monomers is achieved, by further addition of at least one monomer, for example, of the starting mixture. This addition can be carried out in various ways, for example, the sudden addition all at once, and further for example, the continuous addition over the entire duration of the polymerization.

4) The polymerization is halted when the desired degree of conversion is achieved. The overall composition of the copolymer is dependent on the conversion. The polymerization can be halted, for example, after having achieved at least 50% conversion, and for instance, after having achieved at least 90% conversion.

5) The possible residual monomeric units can be removed by any known method, such as by evaporation, and by addition of an amount of conventional polymerization initiator, such as peroxide and azo derivatives.

In one aspect of the present disclosure, the agent for controlling the polymerization, as disclosed herein, is chosen from nitroxides of formula (I):

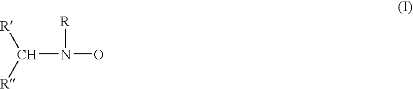

wherein:

R and R′ which may be identical or different, are chosen from linear and branched, saturated alkyl groups comprising 1 to 40 carbon atoms, wherein the alkyl groups comprising 1 to 40 carbon atoms may optionally be substituted by at least one group chosen from —OR₃, —COOR₃ and —NHR₃ (wherein R₃ is chosen from H and linear and branched, saturated alkyl groups comprising 1 to 40 carbon atoms),

wherein it being possible for R and R′ to be connected so as to form a ring.

For example, R and R′ can be chosen from linear and branched alkyl groups comprising 1 to 12 carbon atoms, such as methyl, ethyl, propyl, n-butyl, isobutyl, tert-butyl and pentyl groups. In one aspect of the disclosure, R and R′ are both tert-butyl groups;

R″ is chosen from monovalent groups with a molar mass (Mw) of greater than 16 g/mol, for example, phosphorus-comprising groups of formula:

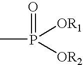

wherein R₁ and R₂, which may be identical or different, are chosen from linear and branched, saturated alkyl groups comprising 1 to 40 carbon atoms, wherein the alkyl groups comprising 1 to 40 carbon atoms may optionally be substituted by at least one group chosen from —OR₃, —COOR₃ and —NHR₃ groups (wherein R₃ is chosen from H and linear and branched, saturated alkyl groups comprising 1 to 40 carbon atoms), and wherein it is possible for R₁ and R₂ to be connected so as to form a ring.

For example, R₁ and R₂ can be chosen from linear and branched alkyl groups comprising 1 to 12 carbon atoms, such as methyl, ethyl, propyl, n-butyl, isobutyl, tert-butyl and pentyl groups. For instance, R₁ and R₂ can both be ethyl groups.

The radical polymerization initiator can be chosen from any conventional polymerization initiator, such as compounds of azo type, for instance, azobisisobutyronitrile, and of peroxide type, such as organic peroxides comprising 6 to 30 carbon atoms, for example, benzoyl peroxide.

According to one aspect of the disclosure, a nitroxide to initiator molar ratio ranging from 1 to 2.5 is observed; further, this ratio can range from 2 to 2.5 when it is believed that one mole of initiator gives rise to two moles of copolymer chains, and can range from 1 to 1.25 for monofunctional initiators.

In another aspect of the present disclosure the radical polymerization initiator is chosen from alkoxyamines of formula (II):

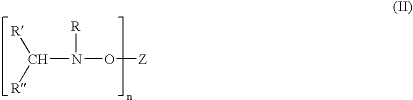

wherein:

R, R′ and R″ have the meanings defined above for the nitroxide of formula (I),

n is an integer of less than or equal to 8, for example, ranging from 1 to 3;

Z is chosen from monovalent and polyvalent radicals, such as styryl, acryloyl and methacryloyl radicals, wherein the alkoxyamines can be chosen in order to simultaneously initiate the polymerization and release the nitroxide that controls the polymerization.

A nitroxide of formula (I) can also be added to the alkoxyamine of formula (II) in an amount ranging from 0 to 20 mol %, relative to the moles of alkoxyamine functional groups (one mole of polyvalent alkoxyamine contributes a number of alkoxyamine functional groups proportional to its valency), so as to improve the quality of the polymerization control.

A person of ordinary skill in the art will know how to choose the initiator according to the present disclosure. Thus, a monofunctional initiator will result in asymmetric chains, whereas a polyfunctional initiator will result in macromolecules having a symmetry starting from a core.

The at least one film-forming gradient copolymer as disclosed herein can be present in the cosmetic varnish compositions in an amount ranging from 0.1 to 60% by weight, relative to the total weight of the composition, for example, from 0.2 to 40% by weight, relative to the total weight of the composition, such as from 1 to 35% by weight, relative to the total weight of the composition, and further for example, from 5 to 30% by weight, relative to the total weight of the composition.

The at least one film-forming gradient copolymer can be present in the composition in dissolved form, for example dissolved in water and/or organic solvents, or else in the form of an aqueous or organic dispersion.

For the purposes of the present disclosure, a polymer is considered “soluble” when it forms, at 1% by weight, a clear solution at 25° C.

For the purposes of the present disclosure, a polymer is considered “dispersible” when it forms, at 1% by weight, at 25° C., a stable suspension of fine particles, such as generally spherical particles, for instance, the mean size of the particles can be less than 1 micron, for example ranging from 5 to 400 nm, and further for example, from 10 to 250 nm, measured by light scattering.

Additionally, it is possible to prepare aqueous and/or organic solutions, and dispersions of the copolymer, by directly mixing the copolymer with water and/or the organic solvent, optionally while heating.

When the gradient copolymer comprises at least one hydrophilic monomeric unit, for example, an aqueous solution or dispersion can be prepared by dissolving the copolymer in an organic solvent having a lower boiling point than water (for example, acetone and methyl ethyl ketone), at solids content ranging from 20 to 90% by weight, relative to the total weight of the composition.

When the hydrophilic monomeric units are chosen from acid types, a solution, for example at least 1 M, of base, such as a hydroxonium ion (OH⁻) salts, amines (such as ammonia), carbonate (CO₃²⁻) salts, and hydrogencarbonate (HCO₃⁻) salts, or of organic neutralizing agent can be added to the organic solution. When the hydrophilic monomeric units are chosen from amine types, a solution, for example at least 1 M, of acid can be added. Water is then added to the solution with vigorous stirring in an amount such that the level of solid obtained ranges from 1 to 80% by weight, relative to the total weight of the composition. The water can optionally be replaced by an aqueous/alcoholic mixture in an amount ranging from 99/1 to 50/50. The solvent is evaporated while stirring the solution at 100° C. Concentration of the solution is continued until the desired level of solid is obtained.

The rate of loss of weight of the nail varnish film is measured according to the protocol described below.

A layer of the composition is deposited on a metal (such as steel) support in order to obtain, after drying at 23±2° C. and at 55+5% relative humidity for 24 hours, a film having a thickness of approximately 100 μm. The film is subjected to abrasion for 60 minutes using the Taber abrasion tester (reference 5130 Abraser), equipped with two abrasive wheels, sold under the name CS10F by Taber Industries, wherein a force of 2.5N is applied to each wheel. During the abrasion operation, the rate of the loss of weight of film is measured over the period of 60 minutes.

The film of nail varnish disclosed herein exhibits a rate of loss of weight of less than 1 mg/minute, for example less than 0.8 mg/minute, such as less than 0.6 mg/minute, and further for example, less than 0.4 mg/minute.

The film of nail varnish exhibits a loss in gloss, after abrading for 10 seconds with the Taber abrasion tester described above, of less than or equal to 14%, for example, less than or equal to 12%, such as less than or equal to 8%. The loss in gloss corresponds to the ratio (Bo−B)/Bo in %, wherein B is the gloss of the film after abrasion, and Bo is the gloss of the film before abrasion. The gloss of the film is measured using a BYK-Gardner glossmeter at a light beam angle of 600, and at 1 hour after abrasion for B.

According to one aspect of the disclosure, the nail varnish composition as disclosed herein, is capable of forming a film having a Young's modulus ranging from 10 to 200 MPa, such as ranging from 10 to 100 MPa, for example, ranging from 10 to 50 MPa.

The mechanical properties of the nail varnish film are measured under monotonic tensile conditions according to the standard ASTM Standards, volume 06.01 D 2370-92, “Standard Test Method for Tensile Properties of Organic Coatings.” A test specimen is cut from a free film having a thickness of 150±50 μm obtained after drying, at 23+2° C. and 55±5% relative humidity for 48 hours, a layer of nail varnish deposited on a Teflon® matrix. The test specimen is of dumbbell shape, with a working length of 33 mm and a working width of 6 mm. The cross section (s) of the test specimen is then defined as: s=width×thickness (mm²); this cross section (s) will be used for the calculation of the stress.

The tests are carried out on a tensile testing device equipped with an optical extensometer for measuring the displacement, which is sold under the name Zwick Z010. The measurements are carried out under the same temperature and humidity conditions as for the drying, that is to say at a temperature of 23±2° C. and a relative humidity of 55±5%. The test specimens are drawn at a rate of displacement of 500 mm/min. A rate of displacement is therefore imposed, and the length (L) of the test specimen and the force (F) necessary to impose this length are measured simultaneously. The length (L) is measured with an optical extensometer using adhesive discs placed on the dumbbell test specimen. The initial distance between these 2 discs defines the working length Lo used to calculate the deformation ε=(L/Lo)×100, expressed in %.

A curve of stress σ (=F/s) as a function of the deformation ε is thus obtained, the test being carried out up to failure of the test specimen. The Young's modulus (modulus of elasticity), expressed in MPa, corresponds to the slope of the linear part of the curve σ=f(ε) (beginning of the test).

According to still another aspect of the present disclosure, the at least one film-forming gradient copolymer is insoluble in water at 25° C., that is to say soluble to less than 1% by weight in water at 25° C. For example, the at least one film-forming gradient copolymer can have a solubility of less than 1% by weight, relative to the total weight of the composition. According to yet another aspect of the present disclosure, the at least one film-forming gradient copolymer is soluble in organic solvents, such as ethyl acetate, butyl acetate and methyl acetate. For example, the at least one film-forming gradient copolymer can have a solubility of greater than 90% by weight at 25° C., relative to the total weight of the composition.

The cosmetic compositions according to the invention comprise, in addition to the said copolymers, a cosmetically acceptable medium, for example, a medium compatible with the nails.

The composition can optionally further comprise at least one additional film-forming polymer chosen, for example, from synthetic polymers of radical type; synthetic polymers of polycondensate type; polymers of natural origin and their blends, such as acrylic polymers; polyurethanes; polyesters; polyamides; polyureas; and cellulose polymers, such as nitrocellulose. Non-limiting mention may also be made of resins, such as sulphonamide resins, alkyd resins and cellulose esters, such as cellulose acetate/butyrate, cellulose acetate and cellulose acetate/propionate.

The at least one additional film-forming polymer may be present in an amount ranging from 0.01 to 50% by weight, relative to the total weight of the composition, for example, ranging from 1 to 30% by weight, relative to the total weight of the composition.

The nail varnish composition according to the present disclosure can also optionally further comprise an additional agent which is able to form a film for improving the film-forming properties of the varnish.

The additional agent which is able to form a film can be chosen from any compound known to a person of ordinary skill in the art as being capable of fulfilling the desired role, for example from plasticizers.

Non-limiting examples of plasticizers, include:

citrates, such as triethyl citrate, tributyl citrate, triethyl acetylcitrate, tributyl acetylcitrate and tri(2-ethylhexyl) acetylcitrate;

phthalates, such as diethyl phthalate, dibutyl phthalate, dioctyl phthalate, dipentyl phthalate and dimethoxyethyl phthalate;

tricresyl phosphate, benzyl benzoate, tributyl phosphate, butyl acetylricinoleate, glyceryl acetylricinoleate, butyl glycolate, tributoxyethyl phosphate, triphenyl phosphate, dibutyl tartrate, camphor, glyceryl triacetate and N-ethyl-o,p-toluenesulphonamide,

and the mixtures thereof.

The plasticizer can be present in the composition in an amount ranging from 0.01 to 10% by weight, relative to the total weight of the composition, for example, ranging from 0.1 to 5% by weight, relative to the total weight of the composition. For example, the plasticizer can be present in the composition according to a ratio by weight of at least one film-forming gradient copolymer to plasticizer, ranging from 1.5 to 3.

The composition according to the present disclosure can comprise an aqueous medium, an aqueous/organic medium or an organic solvent medium. For instance, the composition can comprise an organic solvent medium, which may optionally be anhydrous.

The aqueous medium of the composition can comprise essentially water. The amount of water in the composition can range from 10% to 95% by weight, relative to the total weight of the composition, for instance, from 40% to 90% by weight, relative to the total weight of the composition, such as from 60% to 85% by weight, relative to the total weight of the composition.

When the composition comprises an aqueous medium, the film-forming polymer can be present therein in the form of solid particles dispersed in the aqueous medium or in the dissolved form.

The composition can also comprise an organic solvent, for instance, a water-miscible organic solvent, such as monoalcohols comprising 1 to 5 carbon atoms, glycols comprising 2 to 8 carbon atoms, C₃-C₄ ketones, and C₂-C₄ aldehydes, and for example, in an amount ranging from 0.1% to 15% by weight, relative to the total weight of the composition.

The organic solvent medium or aqueous/organic medium of the composition can comprise at least one organic solvent chosen from:

ketones which are liquid at ambient temperature, such as methyl ethyl ketone, methyl isobutyl ketone, diisobutyl ketone, isophorone, cyclohexanone and acetone;

alcohols which are liquid at ambient temperature, such as ethanol, isopropanol, n-propanol, n-butanol, diacetone alcohol, 2-butoxyethanol and cyclohexanol;

glycols which are liquid at ambient temperature, such as ethylene glycol, propylene glycol, pentylene glycol and glycerol;

propylene glycol ethers which are liquid at ambient temperature, such as propylene glycol monomethyl ether, propylene glycol monomethyl ether acetate and dipropylene glycol mono(n-butyl) ether;

short-chain esters (comprising from 3 to 8 carbon atoms in total), such as ethyl acetate, methyl acetate, propyl acetate, n-butyl acetate and isopentyl acetate;

ethers which are liquid at ambient temperature, such as diethyl ether, dimethyl ether and dichlorodiethyl ether;

alkanes which are liquid at ambient temperature, such as decane, heptane, dodecane, isododecane and cyclohexane;

aromatic cyclic compounds which are liquid at ambient temperature, such as toluene and xylene; and

aldehydes which are liquid at ambient temperature, such as benzaldehyde and acetaldehyde.

The amount of organic solvent in the composition can range from 10% to 95% by weight, relative to the total weight of the composition, for example, from 40% to 90% by weight, relative to the total weight of the composition, such as from 60% to 85% by weight, relative to the total weight of the composition.

The composition can further comprise a thickening agent, for example, used for conferring a consistency on the composition, possibly improving good application of the composition to the nails.

For example, the thickening agent can be a thickener of organic solvents, and can be chosen from hydrophobic silicas, such as those disclosed in document EP-A-898 960 and, for example, sold under the references “Aerosil R812®” by Degussa, “Cab-O-Sil TS-530®”, “Cab-O-Sil TS-610®” and “Cab-O-Sil TS-720®” by Cabot, and “Aerosil R972®” and “Aerosil R974®” by Degussa; clays, such as montmorillonite, stearalkonium hectorite and stearalkonium bentonite; and polysaccharide alkyl ethers (for instance, where the alkyl group comprises 1 to 24 carbon atoms, such as 1 to 10 carbons, for example 1 to 6 carbons, and further for example, 1 to 3 carbons), such as those disclosed in the document EP-A-898 958 and, for example, sold under the names “N-Hance-AG 200®” and “N-Hance AG 50” by Aqualon.

The thickening agent can be present in the composition as disclosed herein, in an amount ranging from 0.05% to 10% by weight, relative to the total weight of the composition, for example, ranging from 0.1% to 3% by weight, relative to the total weight of the composition.

The composition according to the present disclosure can additionally comprise at least one coloring material chosen from water-soluble dyes, fat-soluble dyes and pulverulent coloring materials, such as pigments, pearlescence agents and glitters known to a person of ordinary skill in the art.

The at least one coloring material can be present in the composition in an amount ranging from 0.01 to 50% by weight, relative to the total weight of the composition, for example, from 0.05 to 30% by weight, relative to the total weight of the composition, such as from 0.1 to 25% by weight, relative to the total weight of the composition.

The term “pigments” should be understood as meaning white, colored, inorganic, and organic particles of any shape, which are insoluble in the physiological medium, and which are intended to color the composition. The term “pearlescence agents” should be understood as meaning iridescent particles of any shape, for instance, produced by certain molluscs in their shells, and synthesized.

Non-limiting mention may be made, among inorganic pigments, of: titanium dioxide, optionally surface treated; zirconium and cerium oxides; zinc, iron and chromium oxides (wherein the iron oxides are chosen from black, yellow and red iron oxides); manganese violet; ultramarine blue; chromium hydrate; ferric blue; and metal powders, such as aluminium powder and copper powder.

Non-limiting mention may be made, among organic pigments, of: carbon black, pigments of D & C type, and lakes based on cochineal carmine, barium, strontium, calcium and aluminium.

The pearlescent pigments can be chosen from white pearlescent pigments, such as mica covered with titanium oxide, and/or covered with bismuth oxychloride; colored pearlescent pigments, such as titanium oxide-coated mica covered with iron oxides, titanium oxide-coated mica covered with for example, ferric blue, and chromium oxide, and titanium oxide-coated mica covered with an organic pigment of the abovementioned type, and pearlescent pigments based on bismuth oxychloride.

Non-limiting mention may be made, among water-soluble dyes, of the disodium salt of ponceau, the disodium salt of alizarin green, the trisodium salt of amaranth, the disodium salt of tartrazine, the monosodium salt of rhodamine, the disodium salt of fuchsin, xanthophyll and methylene blue.

Non-limiting examples of fat-soluble dyes include, for example, Sudan red, DC Red 17, DC Green 6, β-carotene, soybean oil, Sudan brown, DC Yellow 11, DC Violet 2, DC Orange 5, and quinoline yellow.

The dyes can be present in the composition in an amount ranging from 0.01% to 6% by weight, relative to the total weight of the composition, for instance, ranging from 0.05% to 3% by weight, relative to the total weight of the composition.

The pigments, pearlescence agents and glitters can be present in the composition, for instance in the base and/or surface composition, in an amount ranging from 0.01% to 25% by weight, relative to the total weight of the composition, such as from 0.05% to 15% by weight, relative to the total weight of the composition.

The composition disclosed herein can also comprise ingredients commonly used in cosmetics, such as fillers, spreading agents, wetting agents, dispersing agents, antifoaming agents, preservatives, UV screening agents, active principles, surfactants, moisturizing agents, fragrances, stabilizing agents, antioxidants, vitamins, trace elements, basifying and/or acidifying agents, ceramides, and the mixtures thereof. Of course, a person of ordinary skill in the art will take care to choose the optional additional compounds, and/or their amounts, so that the advantageous properties of the compositions according to the disclosure are not, or not substantially, detrimentally affected by the envisaged addition.

The nail varnish composition can be employed as base for varnishes, as product for making up the nails, as finishing composition, also known as top coat, to be applied to the product for making up the nails, or else as product for the cosmetic care of the nails. These compositions can be applied to human nails and to false nails.

Another aspect of the present disclosure is a cosmetic method for making up or caring for the nails, comprising the application to the nails of a cosmetic composition as defined above.

The disclosure is illustrated in more detail in the following examples. In these examples, the stable nitroxide, known as SG1, of formula:

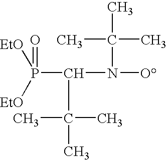

is used as agent for controlling the polymerization.

The polymerization initiators mentioned in the examples are alkoxyamines known as “DIAMS” and “MONAMS” which correspond to the following formulae:

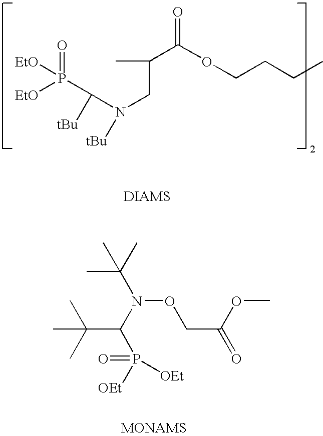

### Example 1

### Bulk Synthesis of Gradient Copolymer

The mixture of reactants was as follows:

| | |
|---|---|
| MONAMS: | 3.0 g |
| SG1: | 0.18 g |
| Ethyl acrylate: | 480 g (i.e. 80% by weight/total weight of monomers) |
| Styrene: | 60 g (i.e. 10% by weight/total weight of monomers) |
| Methacrylic acid: | 60 g (i.e. 10% by weight/total weight of monomers) |

The combined constituents were mixed, in the absence of solvent, under a nitrogen atmosphere and were then heated at a temperature ranging from 110 to 115° C. for 198 minutes. The reaction was halted at a degree of conversion of 60%.

Simulated calculation of the gradient gave the curve below. The theoretical prediction gave 30% incorporation of the (styrene/methacrylic acid) mixture and 70% ethyl acrylate.

This model was validated by monitoring the relative concentrations of the three monomeric units by gas chromatography and NMR analysis of the copolymers.

By these methods, it was found that, at 60% conversion, the final chemical composition of the copolymer was as follows (% by weight): 68.4% ethyl acrylate, 16.1% styrene and 15.5% methacrylic acid according to NMR spectroscopy on the calculated curve (69%).

[image]

The final composition of the copolymer was given by liquid adsorption chromatography (LAC), wherein the plot of the polymer showed the low polydispersity of the chemical composition of the chains.

Measurement of the masses by steric exclusion chromatography gave the following results:

Mn=32 140 g/mol and Mw=51 700 g/mol, resulting in a polydispersity index PI=1.6.

The dispersity in composition (or w) was 1.6.

A diagrammatic representation of the copolymer obtained may be as follows:

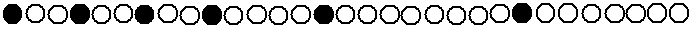

wherein the dark units denote styrene/methacrylic acid sequences and the white units denote ethyl acrylate sequences.

### Example 2

### Bulk Synthesis of Gradient Copolymer

Various copolymers were prepared according to the procedure described in Example 1, starting from the following mixture of reactants:

MONAMS: 3.0 g

SG1: 0.18 g

Styrene: 60 g

Methacrylic acid: 60 g

Acrylate (or mixture of acrylate): 480 g

| | | | Final composition |
|---|---|---|---|
| | | Characteristics | of the |
| | | of the | copolymer (% by |
| Example | Acrylate | copolymer | weight) |
| 2a | Butyl acrylate | Mn = 31 100 g/mol | Styrene: 18 |
| | | Mw = 52 930 g/mol | Methacrylic Ac.: 22 |
| | | Pl = 1.7 | Butyl acrylate: 60 |
| 2b | Methyl acrylate | Mn = 32 750 g/mol | Styrene: 20 |
| | | Mw = 61 470 g/mol | Methacrylic Ac.: 21 |
| | | Pl = 1.88 | Methyl acrylate: 59 |
| 2c | 50/50 Butyl | Mn = 29 690 g/mol | Styrene: 18 |
| | acrylate/ethyl | Mw = 51 630 g/mol | Methacrylic Ac.: 16 |
| | acrylate mixture | Pl = 1.74 | Acrylates: 66 |
| | by weight | | |

### Example 3

### Synthesis in the Presence of Solvent

The same synthesis as in Example 1 was carried out, but in the presence of solvent.

The mixture of reactants was as follows:

MONAMS: 3.43 g

SG1: 0.2 g

Ethyl acrylate: 336 g

Styrene: 42 g

Methacrylic acid: 42 g

Toluene: 180 g

The combined constituents were mixed, in toluene as the solvent, under a nitrogen atmosphere and were then heated at a temperature ranging from 110 to 115° C. for 198 minutes.

The final degree of conversion was 82% and the level of solid obtained was 57.2% by weight.

The following analytical results were determined:

Mn=30 570 g/mol, Mw=50 500 g/mol and Pi=1.65.

The dispersity in composition (or w) was 2.0.

The final composition of the copolymer was given by LAC, which indicated the similarity in composition with the copolymer prepared in Example 1 and the absence of homopolymer in the materials.

### Example 4

### Synthesis in the Presence of Solvent

The synthesis of a new copolymer was carried out according to the process of Example 3, at 120° C. and for 400 minutes, but in a different solvent: methyl ethyl ketone.

The starting composition of the mixture was:

MONAMS: 4.893 g

SG1: 0.2881 g

Ethyl acrylate: 293.8 g

Methyl acrylate: 32.66 g

Styrene: 76.8 g

Methacrylic acid: 76.8 g

Methyl ethyl ketone: 120 g

The final degree of conversion was 99% and the level of solid obtained was 79.9%.

The following analytical results were determined:

Mn=30,500 g/mol

Mw=58,000 g/mol

PI=1.9

The incorporation of the monomers over time was measured by monitoring, by gas chromatography, the levels of residual monomers (in %) over time (in minutes):

| | | time | | | | | |
|---|---|---|---|---|---|---|---|
| | | 0 | 75 | 130 | 190 | 290 | 400 |
| | | Overall conversion | | | | | |
| | | 0 | 16 | 30.5 | 49.5 | 85.4 | 99 |
| Residual monomers | MeA | 5.45 | 5.1 | 3.75 | 3.75 | 1.6 | 0.13 |
| (%) | EA | 48.95 | | | | 17.95 | 1.2 |
| | MAA | 12.8 | 12.15 | 4.6 | 2 | 0.35 | 0.08 |
| | S | 12.8 | 12.46 | 6.7 | 3.92 | 0.15 | 0.007 |

Ethyl acrylate: EA
Methyl acrylate: MeA
Styrene: S
Methacrylic acid: MAA

The total level of residual monomeric units was calculated taking into account the solvent, quantified by the level of solid.

It is noted that each monomer was present throughout the reaction. The gradient determined for each monomeric unit was then be calculated and gave the following curves:

[image]

The final composition of the copolymer was as follows:

ethyl acrylate: 34% by weight

methyl acrylate: 34% by weight

styrene: 16% by weight

methacrylic acid: 16% by weight

### Example 5

The copolymers of Examples 1, 2a and 2c were dissolved in butyl acetate, so as to obtain a solution having a dry matter content of 10% by weight.

The solution obtained was subsequently applied to the nails. After drying, a varnish film is obtained which had the following characteristics, measured according to the protocols described above:

| | Rate of loss of weight | |
|---|---|---|
| | (mg/min) | Loss in gloss (%) |
| Example 1 | 0.3 | 6 ± 2 |
| Example 2a | 0.3 | 6 ± 1 |
| Example 2c | 0.4 | 7 ± 1 |

### Example 6

A nail varnish composition was prepared which had the following composition:

| | |
|---|---|
| Polymer of Example 1 | 23.8 g of active material |
| Butyl acetate | 25.0 g |
| Isopropanol | 10.7 g |
| Hexylene glycol | 2.5 g |
| Pigment (DC Red 7 Lake) | 1 g |
| Modified hectorite | 1.3 g |
| (Bentone ® 27 V from Elementis) | |

After application to the nails, the varnish was judged to exhibit very good properties of hold and of impact strength.

What is claimed is:

1. A nail varnish composition comprising, in a cosmetically acceptable medium, at least one film-forming gradient copolymer comprising at least two different monomeric units, wherein the at least one film-forming gradient copolymer exhibits a mass polydispersity index (PI) of less than or equal to 2.5, and wherein the composition is capable of forming a film exhibiting a rate of loss of weight of less than 1 mg/minute, when the film is subjected to abrasion produced with a Taber abrasion tester at 23° C.

2. The composition according to claim 1, wherein the at least one film-forming gradient copolymer exhibits a mass polydispersity index (PI) ranging from 1.1 to 2.3.

3. The composition according to claim 2, wherein the at least one film-forming gradient copolymer exhibits a mass polydispersity index (PI) ranging from 1.15 to 2.0.

4. The composition according to claim 3, wherein the at least one film-forming gradient copolymer exhibits a mass polydispersity index (PI) ranging from 1.2 to 1.9.

5. The composition according to claim 1, wherein the composition is capable of forming a film exhibiting a rate of loss of weight of less than 0.8 mg/minute, when the film is subjected to abrasion produced with the Taber abrasion tester at 23° C.

6. The composition according to claim 5, wherein the composition is capable of forming a film exhibiting a rate of loss of weight of less than 0.6 mg/minute, when the film is subjected to abrasion produced with the Taber abrasion tester at 23° C.

7. The composition according to claim 6, wherein the composition is capable of forming a film exhibiting a rate of loss of weight of less than 0.4 mg/minute, when the film is subjected to abrasion produced with the Taber abrasion tester at 23° C.

8. The composition according to claim 1, wherein the weight-average molecular mass of the at least one film-forming gradient copolymer ranges from 5,000 g/mol to 1,000,000 g/mol.

9. The composition according to claim 8, wherein the weight-average molecular mass of the at least one film-forming gradient copolymer ranges from 5,500 g/mol to 800,000 g/mol.

10. The composition according to claim 9, wherein the weight-average molecular mass of the at least one film-forming gradient copolymer ranges from 6,000 g/mol to 500,000 g/mol.

11. The composition according to claim 1, wherein the number-average molecular mass of the gradient copolymer ranges from 5,000 g/mol to 1,000,000 g/mol.

12. The composition according to claim 11, wherein the number-average molecular mass of the gradient copolymer ranges from 5,500 g/mol to 800,000 g/mol.

13. The composition according to claim 12, wherein the number-average molecular mass of the gradient copolymer ranges from 6,000 g/mol to 500,000 g/mol.

14. The composition according to claim 1, wherein the at least one film-forming gradient copolymer is such that all the polymer chains have at least one monomeric unit, Mi, for which, whatever the normalized position x on the polymer chain, there is a non-zero probability of encountering the monomeric unit Mi along the chain.

15. The composition according to claim 1, wherein the at least one film-forming gradient copolymer is such that, on the adsorption chromatography (LAC) curve representing the proportion of polymers as a function of the elution volume, the difference (V^{1/2} max−V^{1/2} min) is less than or equal to 3.5, wherein “V^{1/2} min” is the minimum value of the elution volume at mid-height of the curve, and “V^{1/2} max” is the maximum value of the elution volume at mid-height of the curve.

16. The composition according to claim 15, wherein the difference (V^{1/2} max−V^{1/2} min) ranges from 1 and 2.8, wherein “V^{1/2} min” is the minimum value of the elution volume at mid-height of the curve, and “V^{1/2} max” is the maximum value of the elution volume at mid-height of the curve.

17. The composition according to claim 16, wherein the difference (V^{1/2} max−V^{1/2} min) ranges from 1.2 to 2.5, wherein “V^{1/2} min” is the minimum value of the elution volume at mid-height of the curve, and “V^{1/2} max” is the maximum value of the elution volume at mid-height of the curve.

18. The composition according to claim 1, wherein the at least one film-forming gradient copolymer comprises at least two different monomeric units, wherein the at least two different monomeric units are each present in an amount ranging from 1 to 99% by weight, relative to the total weight of the final copolymer.

19. The composition according to claim 18, wherein the at least two different monomeric units are each present in an amount ranging from 2 to 98% by weight, relative to the total weight of the final copolymer.

20. The composition according to claim 19, wherein the at least two different monomeric units are each present in an amount ranging from 5 to 95% by weight, relative to the total weight of the final copolymer.

21. The composition according to claim 1, wherein the at least one film-forming gradient copolymer comprises at least one hydrophilic monomeric unit.

22. The composition according to claim 21, wherein the at least one hydrophilic monomeric unit is present in an amount ranging from 1 to 99% by weight, relative to the total weight of the copolymer.

23. The composition according to claim 22, wherein the at least one hydrophilic monomeric unit is present in an amount ranging from 2 to 70% by weight, relative to the total weight of the copolymer.

24. The composition according to claim 23, wherein the at least one hydrophilic monomeric unit is present in an amount ranging from 3 to 50% by weight, relative to the total weight of the copolymer.

25. The composition according to claim 24, wherein the at least one hydrophilic monomeric unit is present in an amount ranging from 4 to 30% by weight, relative to the total weight of the copolymer.

26. The composition according to claim 25, wherein the at least one hydrophilic monomeric unit is present in an amount ranging from 5 to 25% by weight, relative to the total weight of the copolymer.

27. The composition according to claim 1, wherein the at least one film-forming gradient copolymer comprises at least one monomeric unit whose homopolymer has a Tg of less than or equal to 20° C.

28. The composition according to claim 27, wherein the at least one film-forming gradient copolymer comprises at least one monomeric unit whose homopolymer has a Tg ranging from −150° C. to 20° C.

29. The composition according to claim 28, wherein the at least one film-forming gradient copolymer comprises at least one monomeric unit whose homopolymer has a Tg ranges from −130° C. to 18° C.

30. The composition according to claim 29, wherein the at least one film-forming gradient copolymer comprises at least one monomeric unit whose homopolymer has a Tg ranging from −120° C. to 15° C.

31. The composition according to claim 27, wherein the at least one monomeric unit whose homopolymer has a Tg less than or equal to 20° C. is present in an amount ranging from 1 to 99% by weight, relative to the total weight of the copolymer.

32. The composition according to claim 31, wherein the at least one monomeric unit whose homopolymer has a Tg less than or equal to 20° C. is present in an amount ranging from 20 to 90% by weight, relative to the total weight of the copolymer.

33. The composition according to claim 32, wherein the at least one monomeric unit whose homopolymer has a Tg less than or equal to 20° C. is present in an amount ranging from 30 to 85% by weight, relative to the total weight of the copolymer.

34. The composition according to claim 33, wherein the at least one monomeric unit whose homopolymer has a Tg less than or equal to 20° C. is present in an amount ranging from 50 to 75% by weight, relative to the total weight of the copolymer.

35. The composition according to claim 21, wherein the at least one film-forming gradient copolymer comprises at least one hydrophilic monomeric unit chosen from:
amino(C₁-C₄ alkyl) (meth)acrylate derivatives;
N,N-di(C₁-C₄ alkyl)amino(C₁-C₆ alkyl)(meth)acrylamides;
di(C₁-C₈ alkyl)allylamines;
vinylamines;
vinylpyridines;
and the acid salts and quaternized forms thereof;
ethylenic carboxylic acids;
carboxylic anhydrides with at least one vinyl bond and the salts thereof;
ethylenic sulphonic acids and the salts thereof;
the potassium salts of 3-(acryloyloxy)propanesulphonic acid and the compounds of formula CH₂═CHCOOCH₂OCH₂(OH)CH₂SO₃⁻Na⁺;
amides of unsaturated carboxylic acids;
hydroxyalkyl (meth)acrylates;
(meth)acrylates of polyethylene glycol (5 to 100 EO) and of glycol, which are optionally substituted on their end functional group by a group chosen from alkyl, phosphate, phosphonate and sulphonate groups;
alkoxyalkyl (meth)acrylates;
polysaccharide (meth)acrylates;
vinylamides;
vinyl ethers;
methacrylamidopropoxytrimethylammonium betaine;
N,N-dimethyl-N-methacryloyloxyethyl-N-(3-sulphopropyl)ammonium betaine;
3-methacryloylethoxycarbonylpyridinium;
the compound of formula: 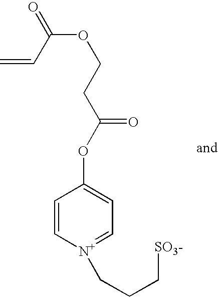
4-vinylpyridiniumsulphopropyl betaine of formula:

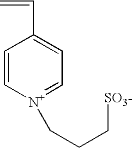

36. The composition according to claim 35, wherein the amino(C₁-C₄ alkyl) (meth)acrylate derivatives are chosen from N,N-di(C₁-C₄ alkyl)amino(C₁-C₆ alkyl) (meth)acrylates.

37. The composition according to claim 36, wherein the N,N-di(C₁-C₄ alkyl)amino(C₁-C₆ alkyl) (meth)acrylates are chosen from N,N-dimethylaminoethyl methacrylate (MADAME) and N,N-diethylaminoethyl methacrylate (DEAMEA).

38. The composition according to claim 35, wherein the N,N-di(C₁-C₄ alkyl)amino(C₁-C₆ alkyl)(meth)acrylamides are chosen from N,N-dimethylacrylamide, N,N-dimethylaminopropylacrylamide (DMAPA) and N,N-dimethylaminopropylmethacrylamide (DMAPMA).

39. The composition according to claim 35, wherein the di(C₁-C₈ alkyl)allylamines are chosen from dimethyldiallylamine.

40. The composition according to claim 35, wherein the vinylpyridines are chosen from 2-vinylpyridine and 4-vinylpyridine.

41. The composition according to claim 35, wherein the ethylenic carboxylic acids are chosen from mono- and dicarboxylic acids.

42. The composition according to claim 41, wherein the mono- and dicarboxylic acids are chosen from acrylic acid, methacrylic acid, crotonic acid, itaconic acid, fumaric acid and maleic acid.

43. The composition according to claim 35, wherein the carboxylic anhydrides with at least one vinyl bond are chosen from maleic anhydride.

44. The composition according to claim 35, wherein the ethylenic sulphonic acids are chosen from styrenesulphonic acid, acrylamidopropanesulphonic acid, vinylbenzoic acid and vinylphosphonic acid.

45. The composition according to claim 35, wherein the amides of unsaturated carboxylic acids are chosen from acrylamide, methacrylamide, and the N-substituted derivatives thereof.

46. The composition according to claim 45, wherein the N-substituted derivatives of acrylamide and methacrylamide are chosen from N-(C₁-C₄ alkyl)(meth)acrylamides and N,N-di(C₁-C₄ alkyl)(meth)acrylamides.

47. The composition according to claim 46, wherein the N-(C₁-C₄ alkyl)(meth)acrylamides are chosen from as N-methylacrylamide.

48. The composition according to claim 46, wherein the N,N-di(C₁-C₄ alkyl)(meth)acrylamides are chosen from N,N-dimethylacrylamide.

49. The composition according to claim 35, wherein the hydroxyalkyl (meth)acrylates are chosen from those whose alkyl group(s) comprise from 2 to 4 carbon atoms.

50. The composition according to claim 49, wherein the hydroxyalkyl (meth)acrylates having alkyl group(s) comprising from 2 to 4 carbon atoms are chosen from hydroxyethyl (meth)acrylate.

51. The composition according to claim 35, wherein the (meth)acrylates of polyethylene glycol (5 to 100 EO) and of glycol which are optionally substituted on their end functional group by a group chosen from alkyl, phosphate, phosphonate and sulphonate groups are chosen from glyceryl acrylate, methoxypolyethylene glycol (8 and 12 EO) (meth)acrylate and hydroxypolyethylene glycol (meth)acrylate.

52. The composition according to claim 35, wherein the alkoxyalkyl (meth)acrylates are chosen from ethoxyethyl (meth)acrylate.

53. The composition according to claim 35, wherein the polysaccharide (meth)acrylates are chosen from sucrose acrylate.

54. The composition according to claim 35, wherein the vinylamides are chosen from vinylacetamides.

55. The composition according to claim 54, wherein the vinylacetamides are chosen from cyclic vinylamides.

56. The composition according to claim 55, wherein the cyclic vinylamides are chosen from vinyllactams.

57. The composition according to claim 56, wherein the vinyllactams are chosen from N-vinylpyrrolidone and N-vinylcaprolactam.

58. The composition according to claim 35, wherein the vinyl ethers are chosen from vinyl methyl ether.

59. The composition according to claim 35, wherein the at least one film-forming gradient copolymer comprises at least one hydrophilic monomeric unit chosen from N,N-dimethylaminoethyl methacrylate (MADAME), acrylic acid, methacrylic acid, crotonic acid, styrenesulphonic acid, acrylamidopropanesulphonic acid, dimethylaminopropylmethacrylamide (DMAPMA), styrenesulphonate, hydroxyethyl acrylate, glyceryl acrylate, ethoxyethyl methacrylate, ethoxyethyl acrylate, methoxypolyethylene glycol (8 and 12 EO) (meth)acrylate, hydroxypolyethylene glycol (meth)acrylate, N-vinylpyrrolidone, N-vinylcaprolactam, acrylamide and N,N-dimethylacrylamide.

60. The composition according to claim 21, wherein the at least one film-forming gradient copolymer comprises at least one monomeric unit chosen from C₁-C₄ alkyl (meth)acrylates which result in the production of (meth)acrylic acid after hydrolysis.

61. The composition according to claim 60, wherein the C₁-C₄ alkyl (meth)acrylates which result in the production of (meth)acrylic acid after hydrolysis are chosen from tert-butyl (meth)acrylate and ethyl (meth)acrylate.

62. The composition according to claim 27, wherein the at least one film-forming gradient copolymer comprises at least one monomeric unit whose homopolymer has a Tg of less than or equal to 20° C., chosen from:
ethylenic hydrocarbons comprising 2 to 10 carbons;
acrylates of formula CH₂═CHCOOR₁, wherein
R₁ is chosen from saturated and unsaturated, linear and branched, alkyl groups comprising 1 to 12 carbons,
provided that R₁ is not chosen from a tert-butyl group, and optionally comprising at least one heteroatom chosen from O, N, S and Si, wherein the alkyl group may optionally be substituted by at least one substituent chosen from hydroxyl groups and halogen atoms chosen from Cl, Br, I and F;
R₁ can also be chosen from —(R″)ₓ—(OC₂H₄)ₙ—OR′″ groups, wherein
x is an integer chosen from 0 and 1,
R″ is chosen from saturated and unsaturated, linear and branched, alkyl groups comprising 1 to 12 carbons,
n is an integer ranging from 5 to 100, and
R′″ is chosen from hydrogen atoms and CH₃;
methacrylates of formula: CH₂═C(CH₃)—COOR₂, wherein
R₂ is chosen from saturated and unsaturated, linear and branched, alkyl groups comprising 3 to 12 carbons, and optionally, at least one heteroatom chosen from O, N, S and Si, and wherein the alkyl group can optionally be substituted by at least one substituent chosen from hydroxyl groups and halogen atoms chosen from Cl, Br, I and F;
R₂ can also be chosen from —(R″)ₙ—(OC₂H₄)ₙ—OR′″ groups, wherein
x is an integer chosen from 0 and 1,
R″ is chosen from saturated and unsaturated, linear and branched, alkyl groups comprising 1 to 12 carbons,
n is an integer ranging from 5 to 100, and
R′″ is chosen from hydrogen atoms and CH₃;
N- and N,N-substituted derivatives of unsaturated C₁-C₁₂ carboxylic acid amides;
vinyl esters of formula: R₃—CO—O—CH═CH₂, wherein R₃ is chosen from linear and branched alkyl groups comprising 2 to 12 carbons; and
vinyl alkyl ethers wherein the alkyl comprises 1 to 12 carbons.

63. The composition according to claim 62, wherein the ethylenic hydrocarbons comprising 2 to 10 carbons are chosen from ethylene, isoprene and butadiene.

64. The composition according to claim 62, wherein the N- and N,N-substituted derivatives of unsaturated C₁-C₁₂ carboxylic acid amides are chosen from N-(C₁-C₁₂ alkyl)(meth)acrylamides.

65. The composition according to claim 62, wherein the N-(C₁-C₁₂ alkyl)(meth)acrylamides are chosen from N-octylacrylamide.

66. The composition according to claim 62, wherein the vinyl esters of formula: R₃—CO—O—CH═CH₂, are chosen from vinyl propionate, vinyl butyrate, vinyl ethylhexanoate, vinyl neononanoate and vinyl neododecanoate;

67. The composition according to claim 62, wherein the vinyl alkyl ethers, wherein the alkyl comprises 1 to 12 carbons, are chosen from methyl vinyl ether and ethyl vinyl ether.

68. The composition according to claim 27, wherein the at least one film-forming gradient copolymer comprises at least one monomeric unit, whose homopolymer has a Tg of less than or equal to 20° C., chosen from:
isoprene and butadiene;
methyl acrylate, ethyl acrylate, isobutyl acrylate, n-butyl acrylate, ethylhexyl acrylate, methoxyethyl acrylate, ethoxyethyl acrylate and hydroxypolyethylene glycol acrylate;
ethoxyethyl methacrylate, hexyl methacrylate, ethylhexyl methacrylate and hydroxypolyethylene glycol methacrylate;
N-(C₆-C₁₂ alkyl)(meth)acrylamides;
vinyl esters of formula: R₃—CO—O—CH═CH₂, wherein R₃ is chosen from linear and branched alkyl groups comprising 6 to 12 carbons.

69. The composition according to claim 68, wherein the N-(C₆-C₁₂ alkyl)(meth)acrylamides are chosen from N-octylacrylamide.

70. The composition according to claim 68, wherein the vinyl esters of formula: R₃—CO—O—CH═CH₂ are chosen from vinyl neononanoate and vinyl neododecanoate.

71. The composition according to claim 1, wherein the at least one film-forming gradient copolymer comprises at least one monomeric unit whose homopolymer has a Tg greater than or equal to 20° C.

72. The composition according to claim 71, wherein the at least one film-forming gradient copolymer comprises at least one monomeric unit whose homopolymer has a Tg greater than or equal to 30° C.

73. The composition according to claim 72, wherein the at least one film-forming gradient copolymer comprises at least one monomeric unit whose homopolymer has a Tg greater than or equal to 50° C.

74. The composition according to claim 71, wherein the at least one monomeric unit whose homopolymer has a Tg greater than or equal to 20° C. is present in the composition in an amount ranging from 1 to 99% by weight, relative to the total weight of the copolymer.

75. The composition according to claim 74, wherein the at least one monomeric unit whose homopolymer has a Tg greater than or equal to 20° C. is present in the composition in an amount ranging from 10 to 80% by weight, relative to the total weight of the copolymer.

76. The composition according to claim 75, wherein the at least one monomeric unit whose homopolymer has a Tg greater than or equal to 20° C. is present in the composition in an amount ranging from 15 to 70% by weight, relative to the total weight of the copolymer.

77. The composition according to claim 76, wherein the at least one monomeric unit whose homopolymer has a Tg greater than or equal to 20° C. is present in the composition in an amount ranging from 25 to 50% by weight, relative to the total weight of the copolymer.

78. The composition according to claim 71, wherein the at least one film-forming gradient copolymer comprises at least one monomeric unit whose homopolymer has a Tg greater than or equal to 20° C. chosen from:
vinyl compounds chosen from groups of formula: CH₂═CH—R₄, wherein
R₄ is chosen from a hydroxyl group; an —NH—C(O)—CH₃ group; an —OC(O)—CH₃ group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group; wherein the cycloalkyl, aryl, aralkyl, heterocyclic and heterocyclylalkyl groups can optionally be substituted by at least one substituent chosen from hydroxyl groups, halogen atoms and linear and branched C₁ to C₄ alkyl groups, wherein the C₁ to C₄ alkyl groups can optionally comprise at least one heteroatoms chosen from O, N, S and P, and further wherein the alkyl groups can optionally comprise at least one substituent chosen from hydroxyl groups, halogens chosen from Cl, Br, I and F atoms, and Si atoms;
acrylates chosen from groups of formula CH₂═CH—COOR₅, wherein
R₅ is chosen from a tert-butyl group; C₃ to C₈ cycloalkyl groups; C₆ to C₂₀ aryl groups; C₇ to C₃₀ aralkyl groups (C₁ to C₄ alkyl groups); 4- to 12-membered heterocyclic groups comprising at least one heteroatom chosen from O, N and S; and heterocyclylalkyl (C₁ to C₄ alkyl) groups;
wherein the cycloalkyl, aryl, aralkyl, heterocyclic and heterocyclylalkyl groups can optionally be substituted by at least one substituent chosen from hydroxyl groups, halogen atoms and linear and branched C₁ to C₄ alkyl groups, wherein the C₁ to C₄ alkyl groups can optionally comprise at least one heteroatom chosen from O, N, S and P, and further wherein, the C₁ to C₄ alkyl groups can optionally be substituted by at least one substituent chosen from hydroxyl groups, halogens chosen from Cl, Br, I and F atoms, and Si atoms; and
methacrylates chosen from groups of formula CH₂═C(CH₃)—COOR₆, wherein
R₆ is chosen from linear and branched C₁ to C₄ alkyl groups, wherein the C₁ to C₄ alkyl groups can optionally be substituted by at least one substituent chosen from hydroxyl groups, halogens chosen from Cl, Br, I and F, and Si atoms; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group;
wherein it the cycloalkyl, aryl, aralkyl, heterocyclic and heterocyclylalkyl groups may optionally be substituted by at least one substituent chosen from hydroxyl groups, halogen atoms, and linear and branched C₁ to C₄ alkyl groups optionally comprising at least one heteroatom chosen from O, N, S and P, and wherein the C₁ to C₄ alkyl groups can be optionally substituted by at least one substituent chosen from hydroxyl groups and halogen atoms chosen from Cl, Br, I and F; and
(meth)acrylamides of formula: CH₂═C(R′)—CO—NR₇R₈, wherein,
R₇ and R₈, which may be identical or different, are chosen from hydrogen atoms and linear and branched alkyl groups comprising 1 to 12 carbon atoms, and
R′ is chosen from hydrogen and methyl.

79. The composition according to claim 78, wherein the heterocyclylalkyl (C₁ to C₄ alkyl) groups are chosen from furfuryl groups.

80. The composition according to claim 78, wherein the linear and branched C₁ to C₄ alkyl groups can be chosen from methyl, ethyl, propyl and isobutyl groups.

81. The composition according to claim 78, wherein the linear and branched alkyl groups comprising 1 to 12 carbon atoms can be chosen from n-butyl, t-butyl, isopropyl, isohexyl, isooctyl and isononyl groups.

82. The composition according to claim 71, wherein the at least one film-forming gradient copolymer comprises at least one monomeric unit, whose homopolymer has a Tg greater than or equal to 20° C., chosen from:
furfuryl acrylate, isobornyl acrylate, tert-butyl acrylate, tert-butylcyclohexyl acrylate and tert-butylbenzyl acrylate;
methyl methacrylate, n-butyl methacrylate, ethyl methacrylate and isobutyl methacrylate;
styrene and styrenesulphonate;
vinyl acetate and vinylcyclohexane.

83. The composition according to claim 1, wherein the at least one film-forming gradient copolymer is present in the composition in an amount ranging from 0.1 to 60% by weight, relative to the total weight of the composition.

84. The composition according to claim 83, wherein the at least one film-forming gradient copolymer is present in the composition in an amount ranging from 0.2 to 40% by weight, relative to the total weight of the composition.

85. The composition according to claim 84, wherein the at least one film-forming gradient copolymer is present in the composition in an amount ranging from 1 to 35% by weight, relative to the total weight of the composition.

86. The composition according to claim 85, wherein the at least one film-forming gradient copolymer is present in the composition in an amount ranging from 5 to 30% by weight, relative to the total weight of the composition.

87. The composition according to claim 1, wherein the at least one film-forming gradient copolymer is present in the composition in a form chosen from the dissolved form and from dispersions.

88. The composition according to claim 87, wherein the dissolved form comprises the at least one film-forming gradient copolymer dissolved in solvents chosen from water and organic solvents.

89. The composition according to claim 88, wherein the dispersions are chosen from aqueous and organic dispersions.

90. The composition according to claim 1, further comprising at least one constituent chosen from additional film-forming polymers, additional agents which are able to form a film, water, organic solvents, thickeners, coloring materials, fillers, spreading agents, wetting agents, dispersing agents, antifoaming agents, preservatives, UV screening agents, active principles, surfactants, moisturizing agents, fragrances, stabilizing agents, antioxidants, vitamins, trace elements, basifying agents, acidifying agents, and ceramides.

91. The composition according to claim 1, wherein the composition is capable of forming a film, and wherein the film has a loss in gloss, after abrading the film for 10 seconds with the Taber abrasion tester, and the gloss being measured 1 hour after abrasion, of less than or equal to 14%.

92. The composition according to claim 91, wherein the composition is capable of forming a film, and wherein the film has a loss in gloss, after abrading the film for 10 seconds with the Taber abrasion tester, and the gloss being measured 1 hour after abrasion, of less than or equal to 12%.

93. The composition according to claim 92, wherein the composition is capable of forming a film, and wherein the film has a loss in gloss, after abrading the film for 10 seconds with the Taber abrasion tester, and the gloss being measured 1 hour after abrasion, of less than or equal to 8%.

94. The composition according to claim 1, wherein the composition is capable of forming a film having a Young's modulus ranging from 10 to 200 MPa.

95. The composition according to claim 94, wherein the composition is capable of forming a film having a Young's modulus ranging from 10 to 100 MPa.

96. The composition according to claim 95, wherein the composition is capable of forming a film having a Young's modulus ranging from 10 to 50 MPa.

97. The composition according to claim 1, wherein the composition is provided in a form chosen from bases for varnishes, products for making up the nails, top coats to be applied to products for making up the nails, and products for the cosmetic care of the nails.

98. A cosmetic method for making up and/or caring for the nails, comprising applying to the nails a cosmetic composition comprising, in a cosmetically acceptable medium, at least one film-forming gradient copolymer comprising at least two different monomeric units, wherein the at least one film-forming gradient copolymer exhibits a mass polydispersity index (PI) of less than or equal to 2.5, and wherein the composition is capable of forming a film exhibiting a rate of loss of weight of less than 1 mg/minute, when the film is subjected to abrasion produced with a Taber abrasion tester at 23° C.
